(19) United States
(12) Patent Application Publication
(10) Pub. No.: US 2011/0212909 A1
(43) Pub. Date: Sep. 1, 2011

(54) Use of Ginsenoside Compound K in the Preparation of a Medicament for the Prevention and Treatment of Atherosclerosis

(75) Inventors: Mengliang Wen, Yunnan (CN); Xiaohui Li, Yunnan (CN); Li Ai, Yunnan (CN); Minggang Li, Yunnan (CN); Gang Du, Yunnan (CN); Jiangyuan Zhao, Yunnan (CN)

(73) Assignee: KUNMING NOVOGINSENG BIOENGINEERING CO., LTD., Kunming, Yunnan (CN)

(21) Appl. No.: 13/125,411

(22) PCT Filed: Oct. 23, 2009
(86) PCT No.: PCT/CN2009/001178

§ 371 Date: Apr. 21, 2011

(30) Foreign Application Priority Data
Oct. 23, 2008 (CN) 200810233470.7

(51) Int. Cl. A61K 31/704; C07J 41/00; A61P 9/10

(52) U.S. Cl. 514/26; 536/5

(57) ABSTRACT

The invention relates to the fields of Chinese drugs and chemical drugs, specifically, drugs comprising ginsenoside compounds, methods of using ginsenoside compound K shown in Formula 1 for prevention and treatment of atherosclerosis and uses of ginsenoside compound K in the preparation of a medicament for prevention and treatment of atherosclerosis.

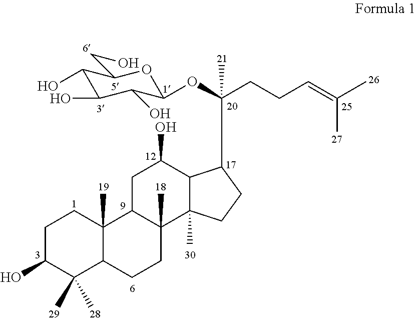

### TECHNICAL FIELD

The invention relates to the fields of Chinese drugs and chemical drugs, specifically, drugs comprising ginsenoside compounds, methods of using ginsenoside compound for prevention and treatment of atherosclerosis and uses of ginsenoside compounds in the preparation of a medicament for prevention and treatment of atherosclerosis.

### BACKGROUND ART

Recently there are about 20 million people died of acute cardiovascular and cerebrovascular events each year in the world, and the mortality rate is increasing every year. And atherosclerosis (AS) is common pathology of the most cardiovascular and cerebrovascular diseases, and induces fatal diseases such as myocardial infarction and cerebral embolism which mortalities are the most high among cardiovascular diseases. Despite various drugs such as statins have been used in clinical, a person skilled in the field has never stopped the research about new drug candidates for prevention and treatment of atherosclerosis.

In Chinese medicine field, prevention and treatment of cardiovascular disease with ginseng and panax notoginseng has a long history, a bulk testing has proved that total panax notoginsenosides can prevent atherosclerosis formation in experimental animals (Yi-Guan Zhang et al, Panax notoginsenosides attenuate atherosclerosis in rats by regulating the blood lipid profile and an anti-inflammatory action. Clin Exp Pharmacol Physiol, 2008, 35 (10): 1238-1244. Gui-Lin Liu et al, Total panax notoginsenosides prevent atherosclerosis in apolipoprotein E-knockout mice: Role of downregulation of CD40 and MMP-9 expression. J Ethnopharmacol, 2009, doi: 10.1016/j.jep.2009.08.014), but the effect of the individual active ingredient of total panax notoginsenosides on atherosclerosis has never been reported.

Though ginsenoside compound K (20-O-β-D-glucopyranosyl 20-(S)-protopanaxadiol, which is called CK for short, structure formula thereof sees Formula 1) belongs to Ginsenosides, and is absent from native Ginseng and Sanchi. In fact, ginsenoside compound K is degradation product of other diol type saponins, such as Rb1, Rb2 and Rc, in human intestinal tract, for example, Rb1 is hardly absorbed in intestinal tract, and is one “natural prodrug”; in fact, ginsenoside compound K is absorbed by human body, and is an entity exerting active effects (Zhou Wei, Zhou Pei, rare ginsenoside compound K studies progress, Acta Pharmaceutica Sinica 2007, 42(9): 917-923). In addition, pharmacokinetics studies showed Ginsenosides K was an active component which entered into blood after conversion.

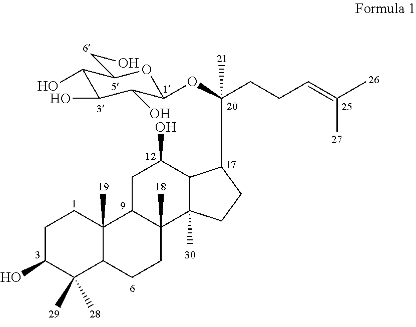

Currently, a person skilled in the art has carried out further studies of ginsenoside compound K in aspect of anti-tumor (Choi H Y et al, A novel ginseng saponin metabolite induces apoptosis and down-regulates fibroblast growth factor receptor 3 in myeloma cells. Int J Oncol, 2003, 23 (4): 1087-1093.), antiinflammation (Shin Y W et al, Effect of ginsenoside Rb1 and compound K in chronic oxazolone-induced mouse dermatitis. Int Immunopharmacol, 2005, 5(7-8): 1183-1191.), antiallergy (Choo M K et al, Antiallergic activity of ginseng and its ginsenosides. Planta Med, 2003, 69 (6): 518-522.), antidiabete mellitus (Han G C et al, Compound K enhances insulin secretion with beneficial metabolic effects in db/db mice. J Agric Food Chem, 2007, 55 (26): 10641-10648.), nerve damage repairement (Jang S et al, Changes of [3H] MK-801, [3H] muscimol and [3H] flunitrazepam binding in rat brain by the prolonged ventricular infusion of transformed ginsenosides. Neurol Res, 2004, 29 (12): 2257-2266.) and the like, but nobody reports effects of ginsenoside compound K as individual component in prevention and treatment of atherosclerosis.

### DESCRIPTION OF INVENTION

In order to provide drug candidates which can be used for prevention and treatment of atherosclerosis, while testing whether ginsenoside compound K can be used in the prevention and treatment of atherosclerosis, applicants has carried out a large number of tests.

Firstly, in vivo toxicity test is conducted in mice by using ginsenoside compound K shown by Formula 1 (20-O-β-D-glucopyranosyl-20 (S)-protopanaxidiol). Then the impact of ginsenoside compound K on total serum cholesterol and cholesterolester content and foam cellularization were detected according to rat abdominal cavity macrophage-derived foam cell formation test (which represented the effects of test compounds on atherosclerosis) known in the art, with pyrrolidine dithiocarbamate as a positive control, and it is found that ginsenoside compound K had the comparative effect of interfering intracellular cholesterol metabolism and anti-foam cellularization to of PDTC of the same concentration.

At the same time, the inventor, conducted a detailed study on mechanism of inhibiting formation of macrophage-derived foam cell in rat abdominal cavity by ginsenoside compound K, effects of ginsenoside compound K on encapsulation protection of lipid in macrophage-derived foam cells, effects of ginsenoside compound K on counter transport of cholesterol in macrophage-derived foam cells in rat abdominal cavity, effects of ginsenoside compound K on blood lipids, AS inflammatory rsponse and plaque stability in the apolipoprotein E gene deficient mice through animal models.

The results showed that ginsenoside compounds K of the invention could be used to prepare drugs for prevention and treatment of AS, and characterized in that ginsenoside compound K could achieve its anti-AS role by the following seven aspects:

(1) Compared with model group, ginsenoside compound K significantly reduced concentrations of serum total cholesterol (TC), triglyceride (TG) and low density lipoprotein cholesterol (LDL-C), and increased concentration of High Density Lipoprotein cholesterol (HDL-C) in apolipoprotein E gene deficient mice (apo E⁻/⁻ mice).

(2) Compared with model group, ginsenoside compound K significantly decreased contents of total cholesterol (TC) and cholesterolester (CE) in macrophage-derived foam cells in rat abdominal cavity.

(3) ginsenoside compound K inhibited formation of apo E⁻/⁻ mice aortic macrophage-derived foam cell and its mechanism of action was as follows:
compared with model group, ginsenoside compound K significantly reduced expression of CD36 mRNA, and reduced uptake of lipid by macrophage;
Compared with model group, ginsenoside compound K significantly reduced expression of perilipin mRNA and protein, and decreased the content of lipid droplets in macrophages;
Compared with model group, ginsenoside compound K significantly upregulated expression of ABCA1 mRNA and LXR α mRNA, and increased counter transport of cholesterol in macrophages.

(4) Compared with model group, ginsenoside compound K significantly reduced concentrations of hs-CRP and sCD40L in serum in apo E⁻/⁻ mice.

(5) Compared with model group, ginsenoside compound K significantly downregulated expression of CD36, perilipin, MMP-9 and NF-κ B mRNA, and upregulated the mRNA expression of ABCA1 and LXRα in apo E⁻/⁻ mice aorta.

(6) Compared with model group, ginsenoside compound K significantly reduced corrected plaque area of apo E⁻/⁻ mice aorta AS (plaque area/vessel cross-sectional area), significantly reduced lipid core area and the corrected lipid core area in AS plaques (lipid core area/plaque area) of apo E⁻/⁻ mice aorta, and significantly increased fibrous cap thickness and corrected collagen area (collagen area/vessel cross section area) of AS plaque of the apo E⁻/⁻ mice aorta, and stabilized AS plaques.

(7) Compared with model group, ginsenoside compound K significantly reduced proportion covered by AS plaque area of aorta in whole aortic intimal area in apo E−/− mice, and reduce the severity of aortic AS pathological change.

It is thereby determined that ginsenoside compound K can be used for prevention and treatment of atherosclerosis, especially aortic, coronary, carotid and cerebral artery type of atherosclerosis, thus the invention is completed.

### DESCRIPTION OF THE FIGURES

FIG. 1 provides effects of a ginsenoside compound K (C-K, 25 μM) and pyrrolidine dithiocarbamate (PDTC, 25 μM) on the foam cell CD36 mRNA expression. a: P<0.05, vs foam cell group; b: P<0.05, vs PDTC. PDTC (25 μM) C-K (25 μM) (n=5).

FIG. 2 provides effects of a ginsenoside compound K (C-K, 25 μM) and pyrrolidine dithiocarbamate (PDTC, 25 μM) on the foam cell perilipin mRNA expression.

a: P<0.05, vs. foam cells group. PDTC (25 μM) C-K (25 μM) (n=5).

FIG. 3 provides effects of a ginsenoside compound K (C-K, 25 μM) and pyrrolidine dithiocarbamate (PDTC, 25 μM) on the protein expression of perilipin in foam cell. a: P<0.01, vs foam cell group PDTC (25 μM) C-K (25 μM) (n=5).

FIG. 4 provides effects of a ginsenoside compound K (C-K, 25 μM) and pyrrolidine dithiocarbamate (PDTC, 25 μM) on the foam cell ABCA1 mRNA expression. a: P<0.05, vs foam cell group. PDTC (25 μM) C-K (25 μM) (n=5).

FIG. 5 provides effects of a ginsenoside compound K (C-K, 25 μM) and pyrrolidine dithiocarbamate (PDTC, 25 μM) on the foam cell LXRα mRNA expression. a: P<0.05, vs foam cell group. PDTC (25 μM) C-K (25 μM) (n=5).

### INVENTION DETAIL

In the present invention, the term “ginsenoside compound K” means a product obtained by microbial transformation of diol-type ginsenosides (Rb1 and Rd) in total panax notoginsenosides, it is the main ingredient which exerts pharmacological activity after absorption of oral Panax, ginseng and preparations thereof into blood. The process by which ginsenoside compound K can be prepared may be obtained from patented method “A method for preparation of rare ginsenoside Compound K by fermentation of notoginsenoside via streptomyces” with the patent number (200710066011. X).

In the present invention, atherosclerosis referred to herein is divided into the following 6 types: (1) atherosclerosis of aorta and its major branches; (2) coronary artery atherosclerosis; (3) carotid arteries and cerebral arteries atherosclerosis; (4) renal artery atherosclerosis; (5) and mesenteric artery atherosclerosis and (6) limb atherosclerosis.

In the present invention, foam cells is used in identifying atherosclerotic effect because numerous studies indicate that formation of macrophage-derived foam cell with a large number of lipid is a characteristic pathological change in early AS, and is core element of AS incidence, wherein the number of foam cells generated has a direct impact on area of plaque lesions, the degree of vascular stenosis, etc. (Kruth H S et al, Macrophage foam cells and atherosclerosis. Front Biosci, 2001, 6: D429-455.

Takahashi K et al, Multifunctional roles of macrophages in the development and progression of atherosclerosis in humans and experimental animals Med Electron Microsc, 2002, 35 (4): 179-203). It has already been commonly accepted in the art that it can be determined whether a drug can be used for prevention and treatment of AS by detecting effects of the drug on foam cells.

Simultaneously, inflammation response is core element for rupture of unstable plaque because inflammation response exists though beginning and development of AS, plaque rupture, and thrombosis. Currently, most AS patient found clinically suffer from intermediate stage and terminal disease, most serious threat of which is complication caused after rupture of unstable plaque. Accordingly, regulation of lipid metabolism, inhibition of inflammation response and stabilization of susceptible plaque has recently become important studies direction for prevention and treatment of AS. And activities of high-sensitive C-Reactive proteins, nucleic factor κB, and matrix metalloproteases and the like are important indications reflecting inflammatory responses and stabilization of susceptible plaque.

mechanism of formation of foam cells predominantly involves three aspects, i.e. uptake, encapsulation protection, and converse efflux of lipid by macrophage: (1) under normal condition, macrophage can not actively phagocytize low density lipoproteins (LDL), and LDL is recognized and phagocytized by receptors corresponding to macrophage only after being oxidized into oxidized low density lipoproteins (ox-LDL). Phagocytosis results in massive deposition of lipid in macrophage, and formation of macrophage-derived foam cells. Endocytosis of lipid by macrophage was accomplished by means of endocytosis mediated by cells surface receptor including two kinds of scavenger receptors A and B which are membrane surface receptors found during studies for mechanism of convention of macrophage into foam cells by human, and exert important action in forming of foam cells and plaque of AS, wherein scavenger receptor cD36 of B type is considered a physiogenic receptor of ox-LDL, and inhibition of CD36 can reduce uptake of lipid by macrophage, and thereby suppressing formation of foam cells. (2) Lipid exists in vesicula formed by related proteins rather than in free form, after entering cells. Protection of these proteins play very important role in preventing and treating hydrolyses of lipid therein, wherein perilipin is an important protein encapsulating intracellular lipid for protection. Expression of perilipin in foam cells has direct relationship with deposition of lipid on artery wall, i.e. forming of atherosclerosis, and the relationship lies in encapsulation protection action of lipid droplets in macrophage-derived foam cells by perilipin, as a result, lowering expression of perilipin mRNA and proteins can reduce amounts of lipid droplets in macrophage, and suppress formation of foam cells. (3) Cells have a set of system for extracellularly antiporting cholesterol in order to maintain intracellular cholesterol homeostasis. The set of system synergeticly acts and interacts with endocytosis and encapsulation system, exerting important action in maintaining cholesterol metabolic equilibrium in cells, wherein ATP-Binding Cassette A1 (ABCA1)-mediated converse efflux of cholesterol into apoprotein deficient of lipid or without lipid (e.g. apoprotein AI, Apo AI) is a uniport process. Additionally, Liver X Receptor α (LXR α) plays important role in maintaining intracellular cholesterol level stable, which transcription factor directly regulates transcription of various genes in cholesterol transport route, and activation of LXR α can promote expression of gene associated with cholesterol efflux route, and lower intracellular cholesterol amount. ABCA1 is the most key element in cholesterol efflux caused by LXR α. Increasing expression of ABCA1 mRNA and LXR α mRNA can increase antiporting of cholesterol in macrophage, and suppress formation of foam cells.

Formation of AS plaque is a topical and systemic inflammation process. Studies show, plaque stability depends on predominant factors such as size of lipid core, thickness of fibrous cap and its repairing ability and the like which are closely associated with AS inflammation responses. Mechanism causing plaque labile by inflammation predominantly presents two aspects: (1) inflammatory cells can promote deposition of lipid in AS plaque. lipoproteins interact with inflammation responses, which forms various cycle, and thereby maks plaque tend to unstable; (2) interaction of inflammatory medium such as matrix metalloproteases, tumor necrosis factor, interleukin, interferon secreted by inflammatory cells can promote degradation of extracellular matrix, alleviate fibrous cap, or suppress extracellular matrix synthesis, lower repairing ability thereof, and thereby make plaque unstable, and result in plaque susceptible and rupture.

Large scale prospective studies results show that high-sensitive C-Reactive proteins (hs-CRP) is a sensitive marker for systematic inflammation responses, and also is a currently most reliable independent predictive factor for Acute Coronary Syndrome (ACS), and is closely associated with formation and development of AS plaque. And soluble CD40L (soluble CD40 ligand, sCD40L), as a transmembrane protein of II type in tumor necrosis factor (TNF) superfamily, binds with its receptor CD40, and can sequently activate production of adhesive molecule, cytokines, chemotactic factor, matrix metalloproteases in AS plaque, and sCD40L is a serum biochemical marker for predicting labile AS plaque. It was found in studies that compared with stable type angina pectoris patient, hs-CRP and sCD40L levels in serum in unstable type angina pectoris patients are obviously higher than those of stable type angina pectoris patients.

Nuclear factor κ B (NF-B) is a class of protein capable of specifically binding with various gene promoter, or enhancer site, and promoting transcription thereof. NF-κ B and inflammatory cytokines, and medium and protease mediated thereby exert extremely important action in occurrence and development of AS. NF-κ B can act as a marker of plaque rupture, and exert important action in plaque rupture by means of regulating transcription of genes such as interleukin-1 (IL-1), interleukin-6 (IL-6), tumor necrosis factor-α (TNF-α), monocyte chemotactic factor 1 (Monocyte Chemotactic protein-1, MCP-1), tissue factor (TF), intercellular adhesion molecule-1 (ICAM-1), vascular cell adhesion molecule-1 (VCAM-1). Activity of NF-κ B in unstable type angina pectoris patients is obviously higher than that of stable type angina pectoris patients, indicating NF-κ B has a close relationship with plaque rupture.

Matrix metalloproteases (MMPs) is a set of protease superfamily of degradable extracellular matrix which enzymic activity depends on zinc ion, and matrix metalloproteases-9 (MMP-9) is especially associated with unstability of AS plaque, in particular in humeral region of unstable plaque, and activity of MMP-9 obviously increases, and is 3 to 5 times higher than that of stable plaque. MMP-9 can specifically bind with extracellular matrix which forms AS plaque fibrous cap moiety, degrade various types of extracellular collagen and gelatin, make elastin weak and fibrous cap of plaque thin, decrease its action resisting stress, and make plaque easy to break.

Accordingly, it can be determined whether drug has an effect of preventing and treating AS by means of confirming that the drug can interfere regulating lipid metabolism of AS patient, lowering its serum hs-CRP and sCD40L level therein, suppress expression of its NF-ic B and MMP-9 therein, and thereby stablize susceptible plaque.

### EXAMPLES

Following particular Examples are used to illustrate technical effects of this invention, and said Examples only are utilized for illustration, not for limiting protection scope of this invention.

### Examples 1

### Primary Survey for Toxicity

40 Kunming mice (weight 18-22 g) were randomly divided into blank group of 20 mice, and ginsenoside compound K group of 20 mice, wherein male and female mice were fifty-fifty in each group. Fasting was carried out before trial for 16 h, allowing mice to freely drink water. Each mouse in drug group was gavaged with ginsenoside compound K (bought from) suspension according to a dose of 400 mg/kg, and blank group was gavaged equivalent amount of distilled water, with administration carried out once a day. Observation was carried out for 14 days after administration, wherein mice were weighted and animal responses were observed each day. The mice were weighted on fourteenth day of the trial, then were sacrificed and dissected, and tissue change of mice's major organs such as heart, liver, spleen, lung, and kidney were observed.

After administration via gavage, phenomena such as eye closed, lying down appeared in mice, and autonomic activities obviously reduced, appetite descent, feces presented black brown, all above signs restored normal in 2 hours after administration. There was no phenomena such as abnormal crying, tremor, convulsion, dyskinesias, salivation, lacrimation, nose running, respiration difficulty, rapid heartbeat or slow heartbeat, diarrhea, constipation, intestinal tympanites in drug group mice; mice's hair was glossy; No mouse died in 14 days of administration. Blank group and drug group mice gained weight, without statistically differences (P>0.05).

Mice were weighted on fourteenth day of the trial, and then were sacrificed and dissected, mice's hearts, livers, spleens, lungs, kidney, ovaries, uterine, seminal vesicles, prostates, testis, stomachs and intestines were observed by naked eye, and there was no abnormality in each organ. Results demonstrated mice's response to acute toxicity of ginsenoside compound K was not obvious.

### Examples 2

### Effects of Ginsenoside Compound K on Formation of Macrophage-Derived Foam Cells of Rat Abdominal Cavity

1. Experiment animal: SD rats, male, 200-250 g, bought from the third Military Medical University Experiment Animal Center.

2. Reagents: RPMI 1640 culture medium free of phenol red bought from Invitrogen company; LDL (115 mg/mL) bought from Peking Union Medical College; total cholesterol kit and free cholesterol kit bought from Shanghai Mind Bioengineering Limited Company; ginsenoside compound K (hereinafter being called C-K for short. White powders, purity 99%), provided by Kunming Nuowei Jinshen Bioengineering limited company supply (batch number: NTGA070521, details see China invention patent 200710066011. X “a method of preparing rare ginsenoside compound K by streptomycete fermentation of Sanchi saponins”).

3. Preparation of ox-LDL

LDL was placed into PBS comprising 10 μM Cu²⁺, dialysed at 37° C. for 12 h, then was placed into PBS comprising 0.01% EDTA, and dialysed at 4° C. for 24 hours before stopping oxidization, then was subjected to filtration sterilization, and stored for use.

4. Culture of macrophage and establishment of foam cells model (available literature: Jia Yi, Li Xiaohui, Xing Mao, Hei Xuefeng, Liu Ya, He Cuiyao. Studies on affect of various combinations of 3 sorts of monomers in total panax notoginsenosides on formation of mouse macrophage-derived foam cells. Journal of China Pharmacy, 2008, 19 (2), 881-883.)

each rat were intraperitoneally injected with 2 mL RPMI 1640 culture solution free of serum and phenol red, and it was sacrificed by cervical dislocation after 20 minutes, and was soaked in 75% ethanol for 10 minutes, then rat's belly was cut open, and fluids in abdominal cavity were collected, and centrifugated at 750 r/min for 5 minutes, and then cells were collected, and cells concentrations were adjusted to 5×10⁶ mL by using RPMI 1640 culture solution which were free of phenol red and comprised 10% Fetal Calf Serum. 10 pieces of 6 wells plate was added 1 mL cells suspension per well, and was placed into an incubator of 5% CO₂ at 37□, and cultured for two hours, then supernatant was discarded, and nonadhesive cells was washed by using PBS. Original culture medium was discarded. Culture was carried out for 48 hours after adding 5 mL culture medium comprising 20 mg/L ox-LDL, and thereby foam cells were formed.

5. Grouping

Mice were divided into ox-LDL model group, pyrrolidine dithiocarbamate (PDTC) (drugs source: Sigma), control group and C-K interference group. Each group had 5 pieces of plates. Culture was carried out in a incabitor of 5% CO₂ at 37° C. for 48 hours (PDTC control group: 25 μM, C-K interference group: 25 μM; action time: 48 hours).

6. Assay of amount of TC and CE in foam cells

Culture solution was aspirated, and lipid in foam cells was extracted by Folch method (reference: Folch J et al, A simple method for the isolation and purification of total lipides from animal tissues. J Biol Chem, 1957, 226 (1): 497-509), according to direction for use of kit.

7. Statistical Analysis

Data was expressed as x̅±s, and variance analysis was carried out between groups by SPSS 13.0 software.

8. Results

Effects of C-K and PDTC on formation of macrophage-derived foam cells in rat abdominal cavity see Table 1, and results showed that: compared with model group, C-K (25 nM) significantly lowered TC and CE amount in foam cells, relieved foam cellularization, which effects are equivalent to those of PDTC of equivalent concentration.

TABLE 1
effects of PDTC (25 μM) and C-K (25 μM) on TC, CE and CE/TC
in foam cells ( x̅ ± s, n = 5)

| Grouping | TC | CE | CE/TC (%) |
|---|---|---|---|
| ox-LDL model group | 87.20 ± 2.19 | 15.24 ± 2.38 | 59.56 ± 2.96 |
| PDTC (25 μM) control group | 60.81 ± 2.62^{a} | 30.63 ± 3.17^{a} | 50.29 ± 3.54^{a} |
| C-K (25 μM) interference group | 59.72 ± 2.38^{a} | 28.12 ± 3.12^{a} | 47.01 ± 3.80^{a} |

Note:
^{a}means P < 0.05 compared with model group.

### Examples 3

### Action Mechanism of Ginsenoside Compound K in Inhibition of Formation of Macrophage-Derived Foam Cells in Rat Abdominal Cavity

### 1. Effects of Ginsenoside Compound K on Uptake of Lipid in Macrophage-Derived Foam Cells in Rat Abdominal Cavity

(1) Material and reagents (the same as Example 2).

(2) real-time quantitative PCR analysis (a reference method: Zhang Yiguan, Li Xiaohui, Fan Jishan, Zhang Haigang, Li Shuhui, Liao Wenqiang, Pang Yan, Jia Yi. Total panax notoginsenosides suppress atherosclerosis formation in rats by means of antiinflammatory and blood fat regulating action. Progress in Modern Biomedicine. 2007, 27(11): 1601-1607).

(3) Statistical analysis

Data was expressed as x̅±s, variance analysis were carried out between groups by SPSS 13.0 software.

(4) Results

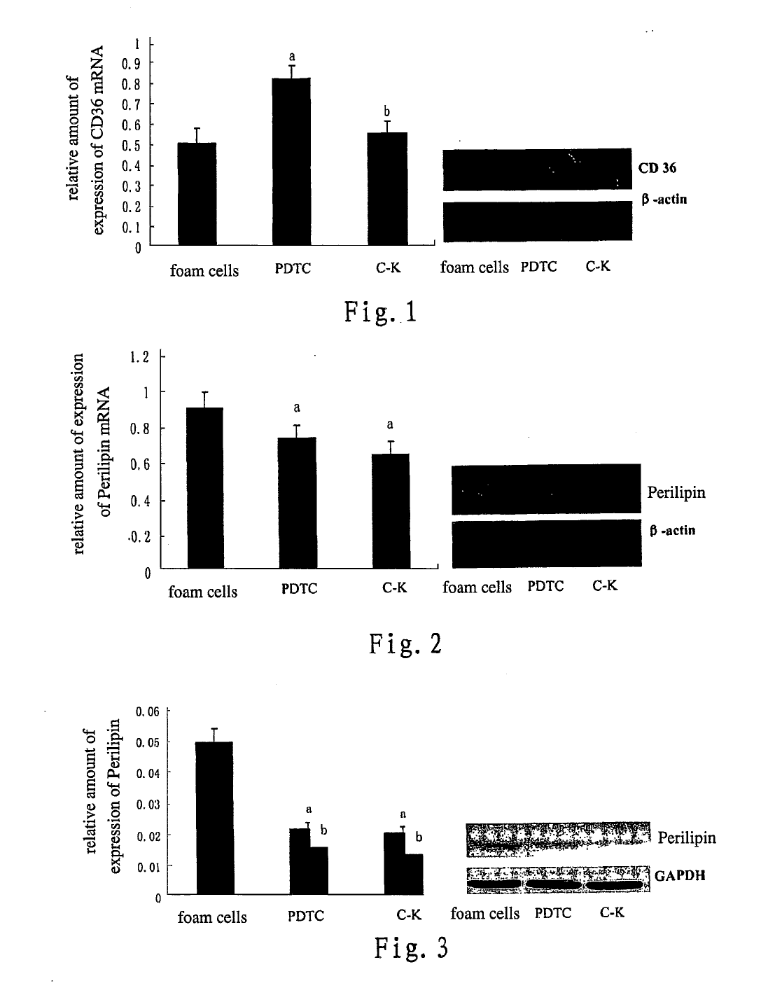

Compared with model group, change of CD36 mRNA expression in foam cells treated by Ginsenoside compound K (25 μM) was not obvious, while positive control drug PDTC (25 μM) could result in increased expression of CD36 mRNA (see FIG. 1).

### 2. Effects of Ginsenoside Compound K on Encapsulation Protection of Lipid in Macrophage-Derived Foam Cells in Rat Abdominal Cavity

(1) Material and reagents (the same as Examples 2).

(2) real-time quantitative PCR analysis (a reference method: Zhang Yiguan, Li Xiaohui, Fan Jishan, Zhang Haigang, Li Shuhui, Liao Wenqiang, Pang Yan, Jia Yi. Total panax notoginsenosides suppress atherosclerosis formation in rats by means of antiinflammatory and blood fat regulating action. Progress in Modern Biomedicine. 2007, 27 (11): 1601-1607).

(3) Statistical analysis

Data was expressed as x̅±s, variance analysis were carried out between groups by SPSS 13.0 software.

(4) Results

Compared with model group, ginsenoside compound K (25 μM) significantly down-regulated expression of perilipin mRNA and proteins, lowered amount of lipid droplets in macrophage (see FIG. 2, FIG. 3), which effects are equivalent to those of PDTC of equivalent concentration.

### 3. Effects of Ginsenoside Compound K on Cholesterol Counter Transport in Macrophage-Derived Foam Cells in Rat Abdominal Cavity

(1) Material and reagents (the same as Examples 2).

(2) real-time quantitative PCR analysis (a reference method: Zhang Yiguan, Li Xiaohui, Fan Jishan, Zhang Haigang, Li Shuhui, Liao Wenqiang, Pang Yan, Jia Yi. Total panax notoginsenosides suppress atherosclerosis formation in rats by means of antiinflammatory and blood fat regulating action. Progress in Modern Biomedicine. 2007, 27 (11): 1601-1607).

(3) Statistical analysis

Data was expressed as x̅±s, variance analysis were carried out between groups by SPSS 13.0 software.

(4) Results

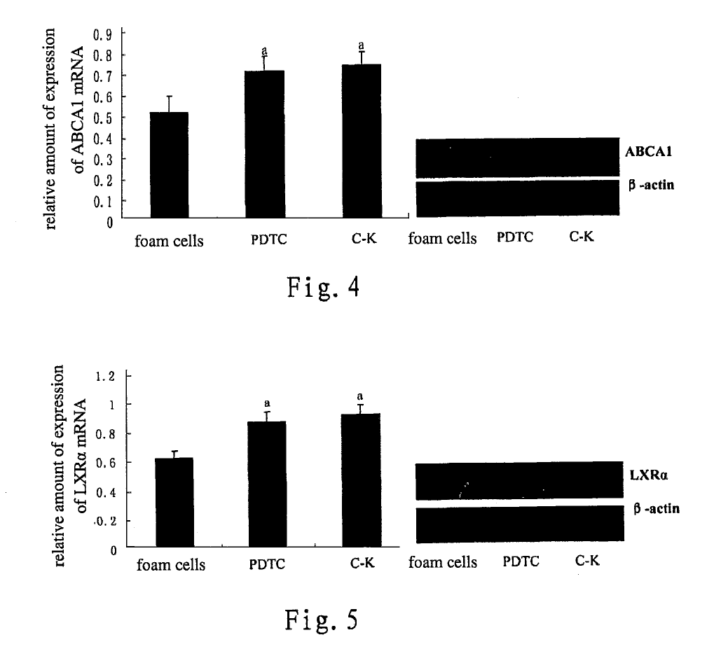

Compared with model group, Ginsenoside compound K (25 μM) significantly up-regulated expression of ABCA1 mRNA, and LXRα mRNA, and increased antiport of cholesterol in macrophage (see FIG. 4, FIG. 5), which effects are equivalent to those of PDTC of equivalent concentration.

### Example 4

### Effects of Ginsenoside Compound K on Blood Fat, AS Inflammatory Responses and Plaque Stability in Apoprotein E Gene Deficient Mouse

### 1. Material and Method

### (1) Animal

80 healthy clean grade C57BL/6J apo E⁻/⁻ mice of 10 weeks old, wherein male and female mice were fifty-fifty, weight 20-22 g, were breed and divided into individual cage in sterile laminar flow rack, allowing mice to freely drink water and get food. Mice were raised for 30 weeks by “Western Diet” (routine mouse feed +0.15% cholesterol +21% fat) high fat feed (⁶⁰cobalt sterilization radiation treatment), with raising conditions being SPF grade, room temperature maintained at 24° C., relative humidity of 50%, and lighting time of 7: 30-19: 30. Animal house was sterilized by ultraviolet lamp once every two days, in order to maintain sterile environment of laminar flow rack (establishment of animal model of atherosclerosis refers to reference: Barish G D, Atkins A R, Downes M, Olson P, Chong L W, Nelson M, Zou Y H, Hwang H S, Kang H J, Curtiss L, Evans R M, Lee C H. PPARδ regulates multiple proinflammatory pathways to suppress atherosclerosis. Proc Natl Acad Sci, 2008, 105 (11): 4271-4276.).

### (2) Animal Grouping and Administration Method

5 mice of apo E⁻/⁻ mice raised for 15 weeks were randomly selected and sacrificed, root of aorta of which was taken, and replication condition in AS model was observed by HE staining. The remaining mice were randomly divided into 5 groups as follows (n=15):

A. model group: vehicle;
B. ginsenoside compound K low dose group: ginsenoside compound K 12.5 mg/kg/day;
C. ginsenoside compound K middle dose group: ginsenoside compound K 25.0 mg/kg/day;
D. ginsenoside compound K high dose group: ginsenoside compound K 50.0 mg/kg/day;
E. simvastatin control group: simvastatin 10.0 mg/kg/day.

Above drugs were firstly solved in DMSO, then suspended in 0.5% carboxymethylcellulose solution, mixed homogeneously, and then were administered via gavage, once a day. Mice were weighted once a week, and ingestion amounts were recorded, and drug doses were adjusted according to weight, interfering for 15 weeks. All the animal were sacrificed in 30^{th} week.

### (3) Animal Sampling

apo E⁻/⁻ mouse serum sample: apo E⁻/⁻ mice were interfered by drug for 15 weeks, then were fasted for 12 hours before sampling. Before being sacrificed, mice were anesthetized by 0.5-1.0 mL 1% Pentobarbital via abdominal cavity, and under sterile condition, 1.5 mL blood form their eye sockets venous plexus was sampled, and centrifugated at 2500 r/min for 10 minutes, and then serum was separated, and frozen and stored at −80° C., for determining blood fat concentrations and inflammatory markers in serum.

Tissues of apo E⁻/⁻ mice were sliced for AS plaque pathology: blood of eye sockets venous plexus of apo E−/− mice was sampled before the mice was sacrificed by cervical dislocation, and aorta was fixed by converse infusion of physiological saline comprising 4% paraformaldehyde into left ventricle, and then whole aorta was cut off from root of aorta to terminal end of abdomen aorta. Root of aorta was taken, and embedded routinely by paraffin, and slices of thickness of 5 μm were continuously cut off from root of aorta, and were subjected separately to HE staining, and MASSON staining, and then were utilized for AS plaque morphological index analysis of aortic valve cross section.

Total RNA sample of root of aorta of apo E⁻/⁻ mouse: Total RNA samples of root of aorta in each group mouse were extracted, and utilized for analysis of gene expression of various lipid metabolic factors, inflammatory factors, and nucleic transcription factors associated with AS plaque development.

Pathological staining: □sequent 2 pieces of slices were picked up from each incisal surface of sequent paraffin slices of aorta root per mouse, and were separately subjected to HE staining, and MASSON staining, and were observed under light microscope. □remaining aorta was stained by Sudan IV, and was observed under light microscope.

### (4) Detecting Index and Detecting Method

blood fat assay: TC, TG, HDL-C, and LDL-C concentrations in serum were determined by applying Olympus Au2700 fully automatic biochemical appearance.
serum inflammatory markers assay: hs-CRP and sCD40L concentrations were determined by applying double antibody sandwich ELISA, in accordance with ELISA kit operating instruction (hs-CRP ELISA kit bought from Herrenberg company, Germany; sCD40L ELISA kit bought from Bender Medsystems company, Austria).
real-time quantitative PCR analysis was carried out on expression of CD36, perilipin, ABCA1, LXRα, MMP-9 and NF-κ B mRNA in apo E⁻/⁻ mouse aorta (a reference method: Zhang Yiguan, Li Xiaohui, Fan Jishan, Zhang Haigang, Li Shuhui, Liao Wenqiang, Pang Yan, Jia Yi. Total panax notoginsenosides suppress atherosclerosis formation in rats by means of antiinflammatory and blood fat regulating action. Progress in Modern Biomedicine. 2007, 27 (11): 1601-1607)₀
morphology index image analysis: When slices were HE stained, under X40 fold ordinary light microscope, artery atherosclerosis plaque area of each incisal surface was determined by “Image Pro Plus 5.0” image analysis software. Plaque area (PA), vessel cross section area (CVA), lipid core area (LCA), and minimal fibrous cap thickness (mFCT) were measured, and corrected plaque area (plaque area/vessel cross section area, PA/CVA) and corrected lipid core area (lipid core area/plaque area, LCA/PA) were calculated, and average of 4 incisal surfaces was taken for each sample. As to Masosn staining, aortic root collagen area (CA) was measured by “Image Pro Plus 5.0” image analysis software, and collagen vessel area ratio (CA/CVA) was calculated. As to Sudan IV staining, entire plaque area of aorta lining endothelium and proportion of plaque area in whole arteria lining endothelium area were calculated via x4 folds light microscope analysis.

### (5) Statistical Analysis

Data was expressed as x̅±s, and variance analysis was carried out between groups by SPSS 13.0 statistical soft ware.

### (6) Results

Effects of ginsenoside compound K (hereinafter being called C-K for short) on blood fat in apo E⁻/⁻ mouse see Table 2. Results showed that: compared with model group, C-K low, middle, and high dose groups all significantly lowered concentrations of TC, TG, and LDL-C in serum of apo E⁻/⁻ mouse, and up-regulated HDL-C concentrations; action of C— K in regulating blood fat is not as good as that of simvastatin.

TABLE 2
effects of C-K on blood fat concentrations in apo E⁻/⁻ mouse (mmol/L, x̅ ± s, n = 15)

| Grouping | TC | TG | HDL-C | LDL-C | TC-HDL/HDL |
|---|---|---|---|---|---|
| Model group | 23.59 ± 3.79^{a} | 2.65 ± 0.39^{a} | 3.79 ± 1.17^{a} | 6.99 ± 1.09^{a} | 5.24 ± 0.36^{a} |
| C-K low dose group | 21.79 ± 4.53^{a} | 2.32 ± 0.41^{a} | 4.08 ± 1.32^{a} | 6.45 ± 1.30^{a} | 4.34 ± 0.16^{a} |
| C-K middle dose group | 21.29 ± 4.44^{a} | 2.28 ± 0.32^{a} | 4.12 ± 1.15^{a} | 6.34 ± 1.22^{a} | 4.17 ± 0.13^{b} |
| C-K high dose group | 20.48 ± 4.34^{b} | 2.19 ± 0.51^{b} | 4.42 ± 1.06^{a} | 6.11 ± 1.18^{a} | 3.63 ± 0.10^{b} |
| simvastatin group | 13.62 ± 4.49^{b} | 1.34 ± 0.46^{b} | 3.80 ± 1.12 | 1.73 ± 1.36^{b} | 2.58 ± 0.18^{b} |

Note:
^{a}means P < 0.05 compared with model group.
^{b}means P < 0.01 compared with model group.

Effects of ginsenoside compound K (hereinafter being called C-K for short) on hs-CRP and sCD40L in apo Er mouse serum see Table 3. Results showed that: compared with model group, C-K low, middle, and high dose groups all significantly lowered concentrations of hs-CRP and sCD40L in apo E⁻/⁻ mouse serum.

TABLE 3
effects of C-K on hs-CRP and sCD40L concentrations in apo E⁻/⁻ mouse serum (ng/mL, x̅ ± s, n = 15)

| | Model group | C-K low dose group | C-K middle dose group | C-K high dose group | simvastatin group |
|---|---|---|---|---|---|
| hs-CRP | 8.13 ± 0.51 | 6.68 ± 0.54^{a} | 6.49 ± 0.36^{a} | 6.14 ± 0.29^{a} | 4.57 ± 0.68^{b} |
| sCD40L | 6.15 ± 0.21 | 5.51 ± 0.35^{a} | 5.34 ± 0.32^{a} | 5.19 ± 0.19^{a} | 4.59 ± 0.37^{b} |

Note:
^{a}means P < 0.05 compared with model group.
^{b}means P < 0.01 compared with model group.

effects of ginsenoside compound K (hereinafter being called C-K for short) on expression of CD36, perilipin, ABCA1, LXRα MMP-9 and NF-κ B mRNA in apo E⁻/⁻ mouse aorta see Table 4. Results showed that: compared with model group, C-K low, middle, and high dose groups all significantly down-regulated expression of CD36, perilipin, MMP-9 and NF-κ B mRNA in apo E⁻/⁻ mouse aorta; C-K low, middle, and high dose groups all significantly up-regulated expression of mRNA of ABCA1, and LXRα in apo E⁻/⁻ mouse aorta.

TABLE 4
effects of C-K on expression of CD36, perilipin, LXRα, ABCA1, MMP-9_{,} and NF-κB mRNA
in apo E⁻/⁻ mouse aorta (relative concentrations) ( x̅ ± s, n = 15)

| Grouping | CD36 | perilipin | LXRα | ABCA1 | MMP-9 | NF-κB |
|---|---|---|---|---|---|---|
| Model group | 1.12 ± 0.13 | 1.08 ± 0.06 | 0.87 ± 0.06 | 0.65 ± 0.18 | 1.15 ± 0.12 | 1.27 ± 0.18 |
| C-K low dose group | 0.54 ± 0.07^{b} | 0.68 ± 0.56^{a} | 1.26 ± 0.05^{b} | 1.08 ± 0.15^{b} | 0.87 ± 0.12^{b} | 0.90 ± 0.19^{b} |
| C-K middle dose group | 0.47 ± 0.08^{b} | 0.60 ± 0.49^{a} | 1.33 ± 0.05^{b} | 1.19 ± 0.18^{b} | 0.78 ± 0.11^{b} | 0.78 ± 0.23^{b} |
| C-K high dose group | 0.31 ± 0.08^{b} | 0.51 ± 0.54^{a} | 1.36 ± 0.06^{b} | 1.34 ± 0.13^{b} | 0.67 ± 0.13^{b} | 0.69 ± 0.16^{b} |
| simvastatin group | 0.77 ± 0.11^{a} | 0.23 ± 1.32^{b} | 0.95 ± 0.07 | 0.78 ± 0.12^{a} | 0.94 ± 0.14^{a} | 0.89 ± 0.16^{b} |

Note:
^{a}means P < 0.05 compared with model group.
^{b}means P < 0.01 compared with model group.

Effects of ginsenoside compound K (hereinafter being called C-K for short) on AS plaque and its stability in apo E⁻/⁻ mouse aorta see Table 5. Results showed that: compared with model group, C-K low, middle, and high dose group significantly lowered AS corrected plaque area (plaque area/vessel cross section area, PA/CVA) in apo E⁻/⁻ mouse aorta, significantly decreased lipid core area and corrected lipid core area (lipid core area/plaque area, LCA/PA) in AS plaque in apo E⁻/⁻ mouse aorta, significantly increased fibrous cap thickness and corrected collagen area (collagen area/vessel cross section area, CA/CVA) of AS plaque in apo E⁻/⁻ mouse aorta, and stabilized AS plaque.

TABLE 5
effects of C-K on AS plaque and components in plaque in apo E⁻/⁻ mouse aorta ( x̅ ± s, n = 15)

| | PA | CVA | LCA | CA | mFCT | PA/CVA | LCA/PA | CA/CVA |
|---|---|---|---|---|---|---|---|---|
| Grouping | (mm²) | | | | (μm) | % | | |
| Model group | 0.90 ± 0.12 | 1.70 ± 0.19 | 0.44 ± 0.05 | 0.39 ± 0.13 | 20.40 ± 3.31 | 53.82 ± 10.02 | 48.28 ± 2.71 | 22.60 ± 5.41 |
| C-K low dose group | 0.78 ± 0.13^{a} | 1.68 ± 0.16 | 0.32 ± 0.05^{b} | 0.56 ± 0.14^{b} | 28.48 ± 1.89^{b} | 46.43 ± 7.38^{a} | 41.03 ± 1.32^{b} | 33.33 ± 4.37^{b} |
| C-K middle dose group | 0.72 ± 0.12^{a} | 1.71 ± 0.14 | 0.29 ± 0.05^{b} | 0.62 ± 0.09^{b} | 30.44 ± 1.75^{b} | 42.26 ± 8.35^{a} | 40.33 ± 1.18^{b} | 36.08 ± 4.82^{b} |
| C-K high dose group | 0.52 ± 0.10^{b} | 1.62 ± 0.12 | 0.21 ± 0.03^{b} | 0.69 ± 0.13^{b} | 30.65 ± 1.37^{b} | 32.29 ± 6.29^{b} | 40.01 ± 1.94^{b} | 42.40 ± 6.74^{b} |
| simvastatin group | 0.74 ± 0.17 | 1.65 ± 0.11 | 0.32 ± 0.04^{b} | 0.57 ± 0.08^{a} | 27.72 ± 2.30^{b} | 44.70 ± 8.44^{a} | 43.39 ± 4.01^{b} | 34.73 ± 4.49^{b} |

Note:
^{a}means P < 0.05 compared with model group.
^{b}means P < 0.01 compared with model group.

Effects of ginsenoside compound K (hereinafter being called C-K for short) on degree of AS pathological change in apo E⁻/⁻ mouse aorta see Table 6. Results showed that: compared with model group, C-K low, middle, and high dose group significantly lowered percentage of AS plaque area in whole arteria lining endothelium area in apo E⁻/⁻ mouse aorta, and relieved degree of AS pathological change in aorta.

TABLE 6
effects of C-K on degree of AS pathological change in apo E⁻/⁻ mouse aorta ( x̅ ± s, n = 15)

| | Model group | C-K low dose group | C-K middle dose group | C-K high dose group | simvastatin group |
|---|---|---|---|---|---|
| plaque area/ | 12.24 ± 0.45 | 9.28 ± 0.39^{b} | 9.54 ± 0.21^{b} | 6.73 ± 0.65^{b} | 6.31 ± 0.19^{b} |
| arteria lining | | | | | |
| endothelium area (%) | | | | | |

Note:
^{b}means P < 0.01 compared with model group.

Atherosclerosis is a chronic pathological process comprising various pathogenic factors, and involving various factor—atherosclerosis is not simply caused by hyperlipemia (JUPITER trial: New Eng J Med, 2008, 359 (21): 2195-2207; ENHANCE trial: New Eng J Med, 2008, 358 (14): 1431-1443); and currently, in the view of prevention and treatment of atherosclerosis, it is commonly accepted that a drug capable of interfering plasma lipoprotein metabolism or intracellular cholesterol metabolism, and interfering inflammatory process, stabilizing plaque (Nature, 2008, 415: 904-913) can be used as a therapy of atherosclerosis. According to trial results of examples, it can be seen that ginsenoside compound K can interfere intracellular cholesterol metabolism and interfere inflammatory process, and stabilize plaque, thus it can be determined that ginsenoside compound K has effects of treating and preventing atherosclerosis.

1-7. (canceled)

8. A method of treating or preventing atherosclerosis, in a subject, comprising administering to the subject a ginsenoside compound K as shown in Formula 1: 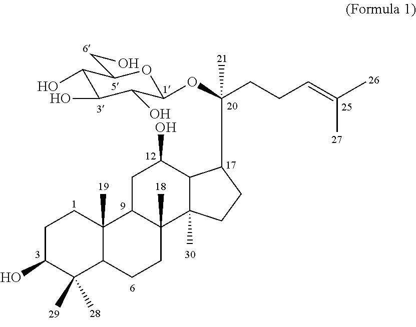
in an amount effective to prevent or treat atherosclerosis in the subject.

9. The method of claim 8, wherein the ginsenoside compound K is administered in an amount effective to interfere with plasma lipoprotein metabolism, interfere with intracellular cholesterol metabolism, interfere with atherosclerosis inflammatory process or stabilize plaque.

10. The method of claim 8, wherein the ginsenoside compound K is administered in an amount effective to interfere with plasma lipoprotein metabolism, interfere with intracellular cholesterol metabolism, interfere with atherosclerosis inflammatory process and stabilize plaque.

11. The method according to claim 8, wherein the ginsenoside compound K is contained within a beverage or a food.

12. The method of claim 8, in which the ginsenoside compound K is administered in an amount effective to downregulate expression of mRNA or protein of perilipin in the subject, and decrease lipid droplets in macrophages of the subject.

13. The method of claim 8, in which the ginsenoside compound K is administered in an amount effective to upregulate expression of ATP-Binding Cassette A1 mRNA and Liver X Receptor α mRNA, and increase counter transport of cholesterol in macrophages of the subject.

14. The method of claim 8, in which the ginsenoside compound K is administered in an amount effective to downregulate expression of mRNA of MMP-9 and NF-κ B in the subject.

15. A method of downregulating expression of mRNA or protein of perilipin, and decreasing lipid droplets in macrophages of a subject, comprising administering to the subject a ginsenoside compound K as shown in Formula 1: [chemical structure]
